(19) United States
(12) Patent Application Publication
(10) Pub. No.: US 2024/0210919 A1
(43) Pub. Date: Jun. 27, 2024

(54) SYSTEM AND METHOD OF IDENTIFYING PATIENT INTERFACE DEVICE USING MASK CLEANING SYSTEM

(71) Applicant: KONINKLIJKE PHILIPS N.V., EINDHOVEN (NL)

(72) Inventors: JONATHAN SAYER GRASHOW, CHESWICK, PA (US); DAAN ANTON VAN DEN ENDE, BREDA (NL)

(21) Appl. No.: 18/544,578

(22) Filed: Dec. 19, 2023

Related U.S. Application Data
(60) Provisional application No. 63/434,493, filed on Dec. 22, 2022.

(51) Int. Cl. G05B 19/4155

(52) U.S. Cl. CPC G05B 19/4155; G05B 2219/35109

(57) ABSTRACT

A system and method of identifying a patient interface device to be used in a pressure support system using a mask cleaning system structured and configured to clean the patient interface device, the mask cleaning system having a controller. The controller is configured to assess the output from a number of sensors as well as the operational parameters of a number of connected components within the mask cleaning system. This assessment enables the controller to obtain values for the operational parameters and the sensors and to further identify the mask coupled to the mask cleaning system. A user may be alerted if the operational parameters signify that the incorrect mask is coupled to the mask cleaning system.

### CROSS-REFERENCE TO RELATED APPLICATIONS

This patent application claims the priority benefit under 35 U.S.C. § 119(e) of U.S. Provisional Application No. 63/434,493, filed on Dec. 22, 2022, the contents of which are herein incorporated by reference.

### BACKGROUND OF THE INVENTION

### 1. Field of the Invention

The disclosed concept relates generally to mask cleaning systems, and, in particular, to a system and method for identifying and alerting users of detected changes in patient interface devices so that appropriate changes to cleaning settings can be made.

### 2. Description of the Related Art

Today, the first line therapy for patients diagnosed with obstructive sleep apnea syndrome (OSAS) after a sleep test is a pressure assisted ventilation support, most often by continuous positive airway pressure (CPAP) therapy. In moderate and severe patients with an AHI>15, the therapy is reimbursed. In mild OSA patients with daytime symptoms, or with chronic and persistent cardiac comorbidities, the PAP therapy is reimbursed for an AHI>5. Reimbursement covers the PAP device as well as periodic resupply of consumable items, such as tubing, headgear, masks and cushions. Additionally, patients may choose to purchase accessories such as mask cleaning or sanitization systems. Several different mask cleaning systems are available, such as ozone-based sanitizers and UV light sanitizers. Alternative consumer mask cleaning systems using detergents, heated fluids, or steam are also possible.

The proper setup of the PAP device including, for instance, pressure settings and fitting of the mask is done by a qualified sleep clinician, most often in an overnight setting at a sleep lab. However, home titration is a possible alternative for certain patients.

Once the correct machine settings and appropriate consumable items are established, the equipment is supplied by a durable medical equipment (DME) supplier and the patient commences therapy. In the event of difficulties with any aspects of the therapy, a consult, optionally followed by re-titration may be performed, which may result in a change in mask type. For instance, a patient may switch from a nasal mask to an oronasal or full-face mask. This change has to be communicated with the DME, who then provides the new mask to the patient.

In addition, patients may also buy a mask out-of-pocket. For instance, if the mask is lost or broken in between reimbursement periods, or if the patient believes that they need a different mask than was advised by the clinician. In either of these cases, the patient may end up using a different mask than was specified by the clinician for their therapy, potentially causing a mismatch in the mask that is used compared to the specified mask. This mismatch potentially has great influence on the therapy because the PAP machine settings are no longer matching the mask being used by the patient.

### SUMMARY OF THE INVENTION

These needs, and others, are met by a system and method of identifying a patient interface device that may be used in a pressure support system using a mask cleaning system. The mask cleaning system may have a controller and may be structured and configured to clean the patient interface device. The method may include the steps of (a) operating the mask cleaning system during a cleaning period to clean the patient interface device, (b) obtaining in the controller one or both of the following: (i) a value for each of a number of operational parameters of the mask cleaning system for the cleaning period, and (ii) a sensor measurement for each of a number of sensors of the mask cleaning system for the cleaning period, and (c) determining an identity of or suspected change of the patient interface device in the controller by one or both of the following: (i) comparing the value for each of the number of operational parameters to operational parameter data stored by the controller, and (ii) comparing the sensor measurement for each of the number of sensors to sensor data stored by the controller. In various embodiments, the number of sensors may include one or more of a pressure sensor, a flow sensor, a temperature sensor, a force sensor, a liquid level sensor, a capacitive sensor, and a magnetic sensor.

Another aspect of the disclosed concept may further include a mask cleaning system structured and configured to clean a patient interface device to be used in a pressure support system. The mask cleaning system may include a main chamber holding apparatus for holding the patient interface device while operating the mask cleaning system during a cleaning period to clean the patient interface device and a controller. The controller may be structured and configured for (a) obtaining one or both of the following: (i) a value for each of a number of operational parameters of the mask cleaning system for the cleaning period, and (ii) a sensor measurement for each of a number of sensors of the mask cleaning system for the cleaning period; and (b) determining an identity of or suspected change of the patient interface device by one or both of the following: (i) comparing the value for each of the number of operational parameters to operational parameter data stored by the controller, and (ii) comparing the sensor measurement for each of the number of sensors to sensor data stored by the controller.

### BRIEF DESCRIPTION OF THE DRAWINGS

A full understanding of the invention can be gained from the following description of the preferred embodiments when read in conjunction with the accompanying drawings in which:

FIG. 1 is a schematic diagram illustrating a system for identifying a patient interface device change, including initial identification at first use, in an ozone pump mask cleaning system while the patient interface device is coupled to the main chamber according to an exemplary embodiment of the disclosed concept;

FIG. 2 is a schematic diagram illustrating a system for identifying a patient interface device change, including initial identification at first use, in mask cleaning system while the patient interface device is retained within a main chamber holding apparatus that includes an integrated capacitive sensor according to an exemplary embodiment of the disclosed concept;

FIG. 3 is a schematic diagram illustrating a system for identifying a patient interface device change, including initial identification at first use, in mask cleaning system while the patient interface device is magnetically coupled to the holding apparatus which includes a resistance sensor integrated therein according to an exemplary embodiment of the disclosed concept; and

FIG. 4 is a flowchart illustrating the overall method according to an exemplary embodiment of the disclosed concept.

### DETAILED DESCRIPTION OF THE INVENTION

As used herein, the terms “cleaning period” and “cleaning operation” refer to a procedure executed by the mask cleaning system to remove chemicals, biological material, debris, and other unwanted contaminants from a mask and/or associated component or system, such as a CPAP apparatus.

As used herein, the term “clean” refers to activities that reduce, remove, sanitize, deactivate, or otherwise minimize the presence of unwanted elements within or around the mask and/or associated components and/or systems.

As used herein, the term “operational parameter” shall refer to a parameter that describes and/or determines the functioning of the mask cleaning system and/or a subsystem of one or more components within the mask cleaning system. For example, the length of time the mask cleaning system requires to reduce contaminants by 10% may be an operational parameter. Likewise, the amount of energy required to increase the temperature of the patient interface device by 5 degrees may be an operational parameter. Other operational parameters may include runtime, pump settings, power consumption, temperature, light intensity, and cleaning fluid type. Further, operational parameters may also refer to a relative value with respect to a baseline (e.g., before during, or after the cleaning period).

As used herein, the term “communicably coupled” shall mean that two or more electrical components are connected in such a way that power, information, or both may be exchanged between the coupled components.

As used herein, the term “operatively coupled” shall mean two or more components are functionally connected, directly or through one or more intermediate parts, such that displacement, manipulation, or actuation of any of the coupled components causes a predefined response in the remaining components.

As used herein, “number” means one or a number greater than one (i.e., a plurality).

The disclosed concept relates to systems and methods for identifying a patient interface device using a mask cleaning system structured and configured to clean the patient interface device. More specifically, the disclosed concept provides, in the various particular embodiments described herein, a mask cleaning system in which changes in the connected patient interface device may be identified by the system based on at least one of a number of operational parameters and/or the output of a number of sensors disposed through the mask cleaning system.

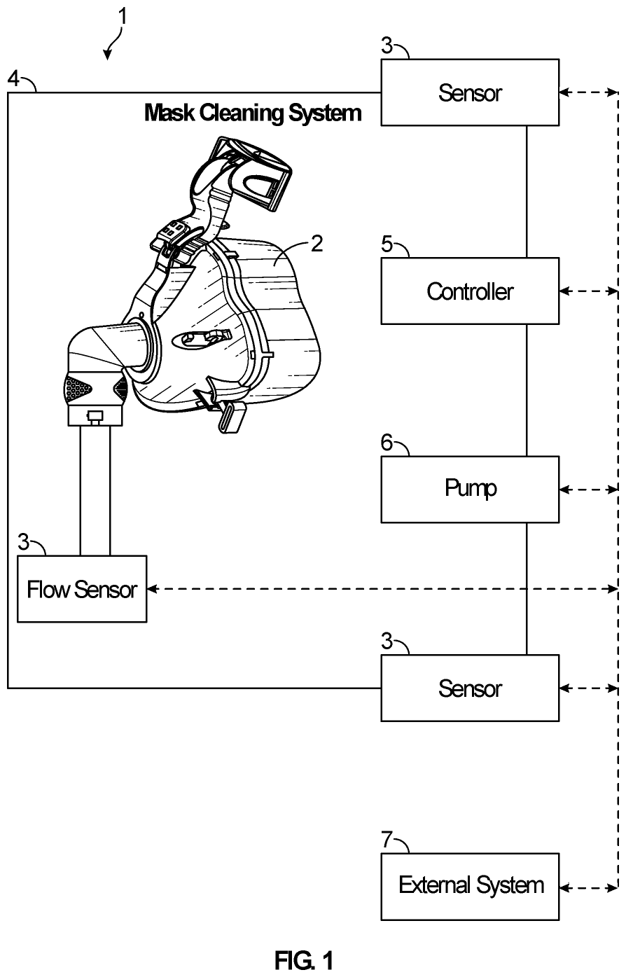

FIG. 1 is a schematic diagram of a mask cleaning system 1 adapted to use a flow of ozone gas generated by mask cleaning system 1 to clean a patient interface device 2 according to one exemplary embodiment of the disclosed concept. Patient interface device 2 in the illustrated exemplary embodiment comprises a full-face mask type mask component. It will be understood, however, that this is meant to be exemplary only, and that other types of mask components are contemplated within the scope of the disclosed concept.

As seen in FIG. 1, mask cleaning system 1 includes a holding apparatus in the form of a main chamber 4 for holding patient interface device 2 during the cleaning process. Mask cleaning system 1 further includes a pump 6 positioned within main chamber 4. Pump 6 is structured and configured for generating the flow of ozone that is used in the cleaning process as described herein. In addition, mask cleaning system 1 also includes number of sensors 3 (e.g., including a flow sensor as shown) for measuring various types of data relating to operation of mask cleaning system 1, such as, without limitation flow, pressure, or temperature. Mask cleaning system 1 further includes a holding apparatus (e.g., in the form of a hose as shown), a controller 5, and a pump 6. Controller 5 is structured and configured for controlling operation of mask cleaning system 1 as described herein. Controller 5 forming part of mask cleaning system 1 may be, for example, a microprocessor, a microcontroller or some other suitable processing device, which includes or is operatively coupled to a memory that provides a storage medium for data and software executable by controller 5 for controlling the operation of mask cleaning system 1. Controller 5 is communicably coupled to each of number of sensors 3 for receiving the data generated by each sensor 3. Further, controller 5 is structured and configured for monitoring various operational parameters of mask cleaning system 1, such as, without limitation, runtime, pump settings, power consumption and/or temperature. Finally, as shown in FIG. 1, mask cleaning system 1 may also be coupled to an external system 7 in the form of an airway pressure support system such as a CPAP apparatus.

In operation, patient interface device 2 is inserted into main chamber 4, and mask cleaning system 1 is coupled to external system 7 by a suitable mechanism such as a fluid conduit. Pump 6 is then activated, and starts generating the flow of ozone within main chamber 4. The flow of ozone within main chamber 4 is then provided to the various internal and external surfaces of patient interface device 2 in order to clean those surfaces. In addition, a portion of the ozone flow is also provided to external system 7 (i.e., the CPAP apparatus in the illustrated embodiment) by way of the associated conduit (i.e., the CPAP apparatus in the illustrated embodiment), in order to clean various internal surfaces thereof, such as the inside of a humidifier forming part of external system 7. As a result, mask cleaning system is able to effectively clean patient interface device 2 and/or external system 7.

In addition, according to a further aspect of the disclosed concept, controller 5 is structured and configured to determine whether a user has changed the patient interface device 2 that is coupled to mask cleaning system 1 since previously using mask cleaning system 1, including initial identification at first use. More specifically, controller 5 is structured and configured for obtaining one or both of the following: (i) a value for each of a number of operational parameters of mask cleaning system 1 for a certain cleaning period, and (ii) a sensor measurement for each of number of sensors 3 for the cleaning period. Controller 5 is also structured and configured for determining an identity of or suspected change of the patient interface device by one or both of the following: (i) comparing the value for each of the number of operational parameters to operational parameter data stored by controller 5 (e.g., in the form of mask profile information), and (ii) comparing the sensor measurement for each of number of sensors 3 to sensor data stored by controller 3 (e.g., in the form of mask profile information). In other words, the current operational parameters of the system as determined in controller 5 and/or the outputs from number of sensors 3 are used to form a current mask profile, which can then be compared to one or more stored mask profiles to determine the identity of patient interface device 2 or a suspected change in the patient interface device 2 that is used with mask cleaning system 1. In a non-limiting embodiment, the current mask profile is automatically generated during the first cleaning period that patient interface device 2 is cleaned by mask cleaning system 1. The initial cleaning period may function as a diagnostic period used to determine the mask type and baseline operational parameters.

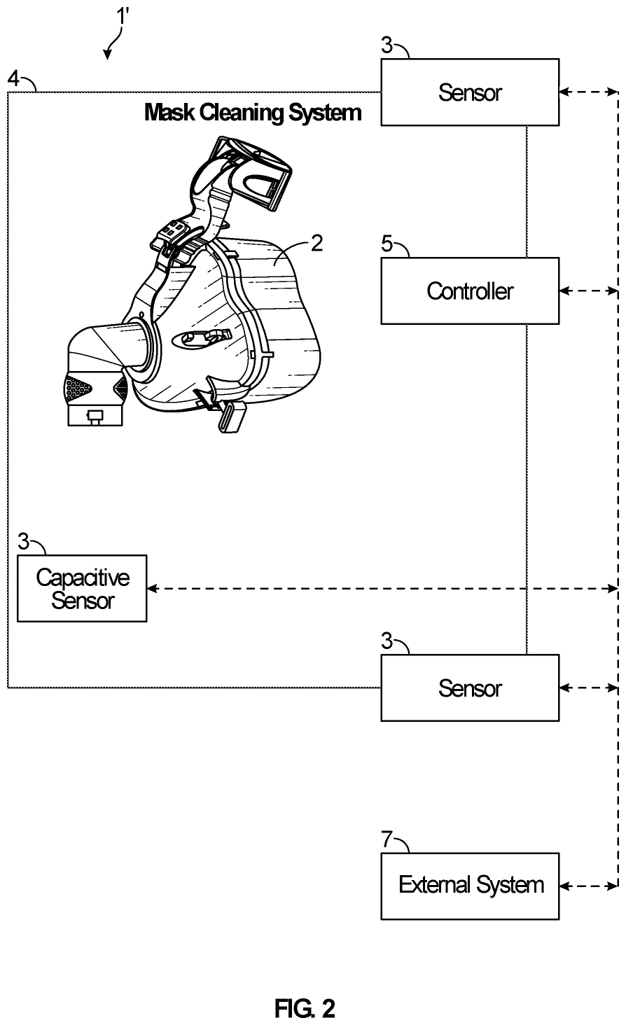

FIG. 2 is a schematic diagram of a mask cleaning system 1′ that is similar to mask cleaning system 1 shown in FIG. 1, and like components are labelled with like reference numerals. Mask cleaning system 1′ may employ any of a number of different cleaning methods/mechanisms, such as, without limitation, a flow of ozone as described above, UV light, detergents/cleaning fluids or steam. In this embodiment, however, chamber 4 of mask cleaning system 1′ is provided with a capacitive sensor 3 integrated therein. Chamber 4 functions as the holding apparatus into which patient interface device 2 is inserted during the cleaning period. Capacitive sensor 3 may be disposed within a chamber 4 floor or other suitable location such as side walls or top cover and is used to detect the presence and/or identity of or change in patient interface device 2 based on the level or degree of disruption of a fringe electromagnetic field that is provided within chamber 4. In particular, the sensor data from capacitive sensor 3 may be compared to a lookup table of capacitive sensor values for known masks. Patient interface device 2 identity may be determined as patient interface device 2 with the closest known capacitive sensor value. This embodiment is operationally similar to the operation of mask cleaning system 1 shown in FIG. 1 and described herein in that after a cleaning operation has been performed, the identity of or suspected change of the patient interface device 2 may be detected by doing one or both of the following: (i) comparing the value for each of a number of operational parameters to operational parameter data stored by controller 5, and (ii) comparing the sensor measurement for capacitive sensor 3 to sensor data stored by the controller.

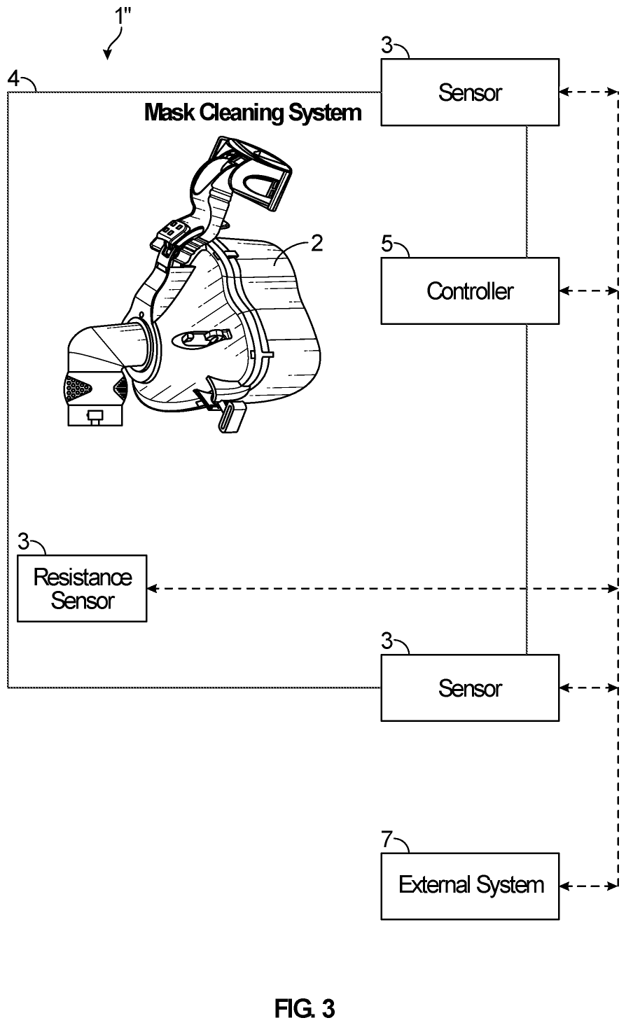

FIG. 3 is a schematic diagram of a mask cleaning system 1″ that is similar to mask cleaning system 1 shown in FIG. 1, and like components are labelled with like reference numerals. Mask cleaning system 1″ may employ any of a number of different cleaning methods/mechanisms, such as, without limitation, a flow of ozone as described above, UV light, detergents/cleaning fluids or steam. In this embodiment, however, chamber 4 includes a primary magnetic coupling device (e.g., a snap connector) that may be mated to a corresponding magnetic coupling device (e.g., a snap connector) on patient interface device 2 during the cleaning period. In an alternative non-limiting embodiment, chamber 4 includes at least one mounting fixture (e.g., including a mechanical connector) that retains patient interface device 2 in a desired orientation. Further, the mounting fixture enables patient interface device 2 to be fluidly and communicably coupled to controller 5 and mask cleaning system 1 such that the mounting fixture provides an additional interface through which to measure mask 2 properties (e.g., operational parameters and sensor data). An advantage of connecting patient interface device 2 in this manner is that patient interface device 2 will be positioned within chamber 4 correctly and the same way for every cleaning period or cycle.

In the embodiment, number of sensors 3 includes a resistance sensor 3 integrated into the primary magnetic coupling device. Resistance sensor 3 is able to facilitate identifying patient interface device 2 by enabling controller 5 to compare a current resistance profile of patient interface device 2 to stored resistance profiles. In a further non-limiting embodiment, number of sensors 3 may be integrated into the mounting fixture and number of sensors 3 may include one or more of the following: magnetometers, strain sensors, resistive sensors, capacitive sensors, pressure sensors, optical sensors, inductive sensors, radio frequency identification (RFID) transceivers, and proximity sensors. This embodiment is operationally similar to the operation of mask cleaning system 1 shown in FIG. 1 and described herein in that after a cleaning operation has been performed, the identity of or suspected change of the patient interface device 2 may be detected by doing one or both of the following: (i) comparing the value for each of a number of operational parameters to operational parameter data stored by controller 5, and (ii) comparing the sensor measurement for resistance sensor 3 to sensor data stored by the controller.

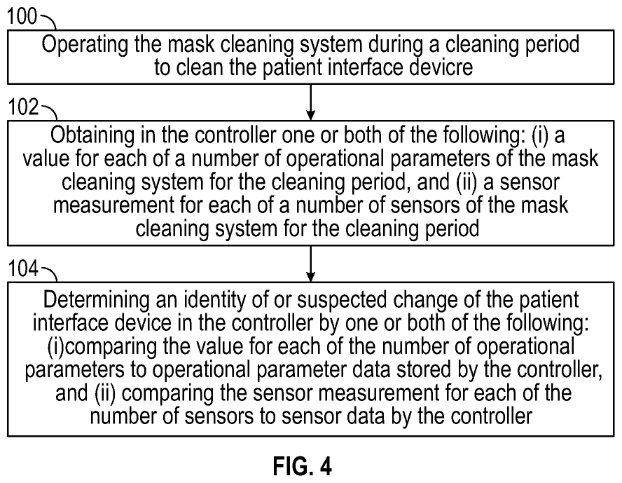

FIG. 4 is a flowchart showing a method of identifying a patient interface device after it has been cleaned using a mask cleaning system according to an exemplary embodiment of the disclosed concept. For illustrative purposes, the method will be described in connection with the mask cleaning system embodiments 1, 1′ and 1″ discussed above, although it will be understood that this is meant to be exemplary only. In addition, in the exemplary embodiment, the method of FIG. 4 is implemented in one or more software routines that are stored and executed by controller 5 forming part of mask cleaning system 1, 1′ or 1″.

Referring to FIG. 4, the method of the disclosed concept begins at step 100 by operating mask cleaning system 1 during a cleaning period to clean patient interface device 2. The method continues at step 102 by obtaining in controller 5 one or both of the following: (i) a value for each of a number of operational parameters of mask cleaning system 1 for the cleaning period, and (ii) a sensor measurement for each of a number of sensors 3 of mask cleaning system 1 for the cleaning period. In a non-limiting embodiment, controller 5 may retain a relative measurement of operational parameters or sensor measurements. For example, a fluid level sensor that first measures fluid level when mask 2 is not yet inserted into chamber 4 and then measures the fluid level when mask 2 is inserted into chamber 4. Accordingly, the operational parameter relating to fluid displacement is a relative value that is proportional to mask 2 volume. In a further non-limiting embodiment, capacitive sensor 3 from FIG. 2 increases mask ID accuracy by gathering a baseline measurement (i.e., empty cleaner) value before the mask 2 is inserted.

The method continues at step 104 by determining an identity of or suspected change of patient interface device 2 in controller 5 by doing one or both of the following: (i) comparing the value for each of the number of operational parameters to operational parameter data stored by controller 5, and (ii) comparing the sensor measurement for each of number of sensors 3 to sensor data stored by controller 5. Controller 5 may analyze multiple datapoints when determining the identity of patient interface device 2 currently attached to mask cleaning system 1. Further, the operational parameter data stored by controller 5 and the sensor data stored by controller 5 may be organized into a number of stored profiles by controller 5. Controller 5 may compare a current mask profile, or identity, to at least one of the number of stored profiles to determine if the current mask profile matches the mask profile of the previous patient interface device 2 coupled to or inserted within main chamber 4. In an embodiment, controller 5 generates an alert if the current mask profile does not match the mask profile of the previous patient interface device 2. The alert may be sent to at least one of a user, a user device, a care provider, a relevant third party, a DME, and external system 7.

In an embodiment, the controller 5 may identify a matching profile from the number of stored profiles when comparing the current mask profile to the stored profile. This matching profile may be designated as the mask identifier (ID). Further, controller 5 may modify operational parameters of mask cleaning system 1 to prevent damage to, or to better clean, patient interface device 2 during subsequent cleaning periods.

In one particular non-limiting exemplary embodiment, 102 may comprise obtaining both of: (i) the value for each of the number of operational parameters of mask cleaning system 1 for the cleaning period, and (ii) the sensor measurement for each of number of sensors 3 of mask cleaning system 1 for the cleaning period. Further, step 104 in this exemplary embodiment may comprise determining the identity of or the suspected change of patient interface device 2 by both of: (i) comparing the value for each of the number of operational parameters to operational parameter data stored by controller 5, and (ii) comparing the sensor measurement for each of number of sensors 3 to sensor data stored by controller 5.

Controller 5 may use this data to determine an appropriate course of action when a difference between current operational parameters and stored operational parameters exceeds predefined thresholds. The number of operational parameters may include a run time of mask cleaning system 1 for the cleaning period. The number of operational parameters may further include a level of power consumption of mask cleaning system 1 for the cleaning period. The number of operational parameters may further include a fluid pressure level or a fluid flow level of mask cleaning system 1 for the cleaning period. The number of operational parameters may further include a temperature level of mask cleaning system 1 for the cleaning period. The number of operational parameters may further include an amount of heat used by mask cleaning system 1 for the cleaning period. The number of operational parameters may further include a number of operational settings of mask cleaning system 1 for the cleaning period.

It is contemplated that aspects of the disclosed concept can be embodied as computer readable codes on a tangible computer readable recording medium. The computer readable recording medium is any data storage device that can store data which can be thereafter read by a computer system. Examples of the computer readable recording medium include read-only memory (ROM), random-access memory (RAM), CD-ROMs, magnetic tapes, floppy disks, and optical data storage devices.

While specific embodiments of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of disclosed concept which is to be given the full breadth of the claims appended and any and all equivalents thereof.

What is claimed is:

1. A method of identifying a patient interface device to be used in a pressure support system using a mask cleaning system structured and configured to clean the patient interface device, the mask cleaning system having a controller, the method comprising:
(a) operating the mask cleaning system during a cleaning period to clean the patient interface device;
(b) obtaining in the controller one or both of the following: (i) a value for each of a number of operational parameters of the mask cleaning system for the cleaning period, and (ii) a sensor measurement for each of a number of sensors of the mask cleaning system for the cleaning period; and
(c) determining an identity of or suspected change of the patient interface device in the controller by one or both of the following: (i) comparing the value for each of the number of operational parameters to operational parameter data stored by the controller, and (ii) comparing the sensor measurement for each of the number of sensors to sensor data stored by the controller.

2. The method according to claim 1, wherein the number of operational parameters includes a run time of the mask cleaning system for the cleaning period.

3. The method according to claim 1, wherein the number of operational parameters includes a level of power consumption of the mask cleaning system for the cleaning period.

4. The method according to claim 1, wherein the number of operational parameters includes a fluid pressure level or a fluid flow level of the mask cleaning system for the cleaning period.

5. The method according to claim 1, wherein the number of operational parameters includes a temperature level of the mask cleaning system for the cleaning period.

6. The method according to claim 1, wherein the number of operational parameters includes an amount of heat used by the mask cleaning system for the cleaning period.

7. The method according to claim 1, wherein the number of operational parameters includes a number of operational settings of the mask cleaning system for the cleaning period.

8. The method according to claim 1, wherein the number of sensors includes one or more of a pressure sensor, a flow sensor, a temperature sensor, a force sensor, a liquid level sensor, a capacitive sensor and a magnetic sensor.

9. The method according to claim 1, wherein (b) comprises obtaining both of: (i) the value for each of the number of operational parameters of the mask cleaning system for the cleaning period, and (ii) the sensor measurement for each of the number of sensors of the mask cleaning system for the cleaning period, and wherein (c) comprises determining the identity of or the suspected change of the patient interface device by both of: (i) comparing the value for each of the number of operational parameters to operational parameter data stored by the controller, and (ii) comparing the sensor measurement for each of the number of sensors to sensor data stored by the controller.

10. A mask cleaning system structured and configured to clean a patient interface device to be used in a pressure support system, comprising:
a main chamber for holding the patient interface device while operating the mask cleaning system during a cleaning period to clean the patient interface device; and
a controller structured and configured for:
(a) obtaining one or both of the following: (i) a value for each of a number of operational parameters of the mask cleaning system for the cleaning period, and (ii) a sensor measurement for each of a number of sensors of the mask cleaning system for the cleaning period; and
(b) determining an identity of or suspected change of the patient interface device by one or both of the following: (i) comparing the value for each of the number of operational parameters to operational parameter data stored by the controller, and (ii) comparing the sensor measurement for each of the number of sensors to sensor data stored by the controller.

11. The mask cleaning system according to claim 10, wherein the number of operational parameters includes a run time of the mask cleaning system for the cleaning period.

12. The mask cleaning system according to claim 10, wherein the number of operational parameters includes a level of power consumption of the mask cleaning system for the cleaning period.

13. The mask cleaning system according to claim 10, wherein the number of operational parameters includes a fluid pressure level or a fluid flow level of the mask cleaning system for the cleaning period.

14. The mask cleaning system according to claim 10, wherein the number of operational parameters includes a temperature level of the mask cleaning system for the cleaning period.

15. The mask cleaning system according to claim 10, wherein the number of operational parameters includes an amount of heat used by the mask cleaning system for the cleaning period.

16. The mask cleaning system according to claim 10, wherein the number of operational parameters includes a number of operational settings of the mask cleaning system for the cleaning period.

17. The mask cleaning system according to claim 10, wherein the number of sensors includes one or more of a pressure sensor, a flow sensor, a temperature sensor, a force sensor, a liquid level sensor, a resistive sensor, a capacitive sensor and a magnetic sensor.

18. The mask cleaning system according to claim 10, wherein (a) comprises obtaining both of: (i) the value for each of the number of operational parameters of the mask cleaning system for the cleaning period, and (ii) the sensor measurement for each of the number of sensors of the mask cleaning system for the cleaning period, and wherein (b) comprises determining the identity of or the suspected change of the patient interface device by both of: (i) comparing the value for each of the number of operational parameters to operational parameter data stored by the controller, and (ii) comparing the sensor measurement for each of the number of sensors to sensor data stored by the controller.
